(19) United States
(12) Patent Application Publication
(10) Pub. No.: US 2020/0405708 A1
(43) Pub. Date: Dec. 31, 2020

(54) TREATMENT OF HEREDITARY ANGIOEDEMA

(71) Applicant: Attune Pharmaceuticals, Inc., New York, NY (US)

(72) Inventors: Andrew MCDONALD, New York, NY (US); Shawn QIAN, New York, NY (US); Ira KALFUS, New York, NY (US)

(21) Appl. No.: 16/976,640

(22) PCT Filed: Feb. 28, 2019
(86) PCT No.: PCT/IB19/00186

§ 371 Date: Aug. 28, 2020

Related U.S. Application Data
(60) Provisional application No. 62/636,809, filed on Feb. 28, 2018.
(60) Provisional application No. 62/641,144, filed on Mar. 9, 2018.

(51) Int. Cl. A61K 31/4709; A61K 9/00; A61P 7/10

(52) U.S. Cl. CPC A61K 31/4709; A61P 7/10; A61K 9/0053

(57) ABSTRACT

Described herein are compositions comprising a highly selective at plasma kallikrein inhibitor and useful for the treatment of angioedema.

### CROSS REFERENCE TO RELATED APPLICATIONS

This application claims the benefit of U.S. Provisional Application No. 62/636,809, filed Feb. 28, 2018, and U.S. Provisional Application No. 62/641,144, filed Mar. 9, 2018, each of which is incorporated by reference in the disclosure of this application.

### BACKGROUND

A need exists in the medicinal arts for the effective treatment of diseases and disorders related to the vascular system. Such diseases and disorders include, but are not limited to, angioedema, macular edema and brain edema.

### SUMMARY OF THE DISCLOSURE

In an aspect, provided herein is N-((6-amino-2,4-dimethylpyridin-3-yl)methyl)-2-((3-chloroquinolin-6-yl)methyl)isonicotinamide, or a pharmaceutically acceptable salt thereof, for use in a method of treatment of the human or animal body.

In another aspect, provided herein is a method of treating angioedema in a patient in need thereof, comprising administration of a composition comprising N-((6-amino-2,4-dimethylpyridin-3-yl)methyl)-2-((3-chloroquinolin-6-yl)methyl)isonicotinamide, or a pharmaceutically acceptable salt thereof.

In some embodiments, the angioedema is hereditary angioedema.

In some embodiments, the composition is administered daily. In some embodiments, the composition is administered once or twice per day. In some embodiments, the N-((6-amino-2,4-dimethylpyridin-3-yl)methyl)-2-((3-chloroquinolin-6-yl)methyl)isonicotinamide is administered twice per day. In some embodiments, the N-((6-amino-2,4-dimethylpyridin-3-yl)methyl)-2-((3-chloroquinolin-6-yl)methyl)isonicotinamide is administered once per day.

In some embodiments, the composition is administered orally.

In some embodiments, the N-((6-amino-2,4-dimethylpyridin-3-yl)methyl)-2-((3-chloroquinolin-6-yl)methyl)isonicotinamide is administered in an amount of from about 300 mg/day to about 800 mg/day.

In some embodiments, the N-((6-amino-2,4-dimethylpyridin-3-yl)methyl)-2-((3-chloroquinolin-6-yl)methyl)isonicotinamide is administered in an amount of about 300 mg/day, about 350 mg/day, about 400 mg/day, about 450 mg/day, about 500 mg/day, about 600 mg/day, about 650 mg/day, about 700 mg/day, about 750 mg/day, or about 800 mg/day. In some embodiments, the N-((6-amino-2,4-dimethylpyridin-3-yl)methyl)-2-((3-chloroquinolin-6-yl)methyl)isonicotinamide is administered in an amount of about 400 mg/day, about 450 mg/day, about 500 mg/day, about 600 mg/day, about 650 mg/day, about 700 mg/day, about 750 mg/day, or about 800 mg/day. In some embodiments, the N-((6-amino-2,4-dimethylpyridin-3-yl)methyl)-2-((3-chloroquinolin-6-yl)methyl)isonicotinamide is administered in an amount of about 400 mg/day. In some embodiments, the N-((6-amino-2,4-dimethylpyridin-3-yl)methyl)-2-((3-chloroquinolin-6-yl)methyl)isonicotinamide is administered in an amount of about 450 mg/day. In some embodiments, the N-((6-amino-2,4-dimethylpyridin-3-yl)methyl)-2-((3-chloroquinolin-6-yl)methyl)isonicotinamide is administered in an amount of about 500 mg/day. In some embodiments, the N-((6-amino-2,4-dimethylpyridin-3-yl)methyl)-2-((3-chloroquinolin-6-yl)methyl)isonicotinamide is administered in an amount of about 550 mg/day. In some embodiments, the N-((6-amino-2,4-dimethylpyridin-3-yl)methyl)-2-((3-chloroquinolin-6-yl)methyl)isonicotinamide is administered in an amount of about 600 mg/day. In some embodiments, the N-((6-amino-2,4-dimethylpyridin-3-yl)methyl)-2-((3-chloroquinolin-6-yl)methyl)isonicotinamide is administered in an amount of about 650 mg/day. In some embodiments, the N-((6-amino-2,4-dimethylpyridin-3-yl)methyl)-2-((3-chloroquinolin-6-yl)methyl)isonicotinamide is administered in an amount of about 700 mg/day. In some embodiments, the N-((6-amino-2,4-dimethylpyridin-3-yl)methyl)-2-((3-chloroquinolin-6-yl)methyl)isonicotinamide is administered in an amount of about 750 mg/day. In some embodiments, the N-((6-amino-2,4-dimethylpyridin-3-yl)methyl)-2-((3-chloroquinolin-6-yl)methyl)isonicotinamide is administered in an amount of about 800 mg/day.

In some embodiments, the composition is formulated for immediate release.

In some embodiments, the composition is formulated for as a tablet or capsule.

In some embodiments, the composition further comprises at least one pharmaceutically acceptable excipient.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

FIG. 1. A flow-chart depicting an overview of hereditary angioedema (HAE) and C1-INH pathway-specific treatment options.

FIG. 2. A table demonstrating the selectivity of Compound A versus other serine proteases.

FIGS. 3A-3B. Graphic representations of the potency of Compound A and C1-INH at inhibiting plasma kallikrein in biochemical inhibition (FIG. 3A) and contact activation (FIG. 3B) assays.

FIG. 4. Graphic depiction of the pharmacokinetic exposure (plasma concentration (ng/mL)) of Compound A after a single oral dosing at 15 mg/kg in monkeys. * Approximately 50 mg/animal (400 mg human equivalent dose). ** EC₅₀ and EC₉₀ derived from contact activation assay inhibition.

FIG. 5. Graphic depiction of mean±S.D. plasma concentration after single oral dosing of Compound A at 15 mg/kg in monkeys.

FIG. 6. A table demonstrating cytochrome P450 inhibition—drug concentration required for cytochrome P450 (CYP) inhibition.

FIG. 7. A table showing metabolism and pharmacokinetics results of single oral dosing of Compound A. Metabolism study was conducted with radiolabeled [¹⁴C] Compound A.

FIG. 8. Graphic representation of 28-day repeat exposure in monkey toxicology study (NOAEL (100 mg/kg/day) at Day 28.

FIG. 9. A table showing safety pharmacology of Compound A single dose in rat and monkey.

FIG. 10. A table showing toxicology/genotoxicity of Compound A.

FIG. 11. Graphic depiction of mean±S.D. plasma concentration of Compound A 0-48 hours post dose-fasted population (n=36).

FIG. 12. Ln AUC_{inf} of Compound A by Ln Dose-fasted population (n=36, 6 in each dose cohort).

FIG. 13. Ln Cₘₐₓ of Compound A by Ln Dose-fasted population (n=36, 6 in each dose cohort).

### DETAILED DESCRIPTION

### Definitions

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth. When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range. The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features. The abbreviations used herein have their conventional meaning within the chemical and biological arts. The chemical structures and formulae set forth herein are constructed according to the standard rules of chemical valency known in the chemical arts.

“Pharmaceutically acceptable salt” includes both acid and base addition salts. A pharmaceutically acceptable salt of any one of the kallikrein inhibitory compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

As used herein, “treatment” or “treating,” or “palliating” or “ameliorating” are used interchangeably. These terms refer to an approach for obtaining beneficial or desired results including but not limited to therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient is still afflicted with the underlying disorder. For prophylactic benefit, the compositions are, in some embodiments, administered to a patient at risk of developing a particular disease, or to a patient reporting one or more of the physiological symptoms of a disease, even though a diagnosis of this disease has not been made.

### Kallikrein-Kinin System

Modulation of vascular permeability is important in regulating the passage of small molecules or blood cells between blood vessels and surrounding tissues. Vascular permeability depends upon the physiological states of tissues such as during inflammation, changes in blood pressure, and fluctuations in ion and nutrient gradients. The junctions between the endothelial cells that line blood vessels are the immediate controllers of vascular permeability. The strength of these junctions is tightly regulated by the kinin-kallikrein system of polypeptides and enzymes. Abnormalities in the kinin-kallikrein system lead to a range of pathologies including angioedema, macular edema and brain edema. Angioedema is a potentially fatal blood disorder characterized by swelling that may occur in the face, gastrointestinal tract, extremities, genitals and upper airways. Genetic hereditary angioedema attacks result from the unregulated activation of the kallikrein system with uncontrolled increases in vascular permeability. Currently there is a need for agents that are useful for the treatment of angioedema and for agents that inhibit plasma kallikrein.

The kallikrein-kinin system represents a metabolic cascade that, when activated, triggers the release of vasoactive kinins. The kinin-kallikrein system (KKS) consists of serine proteases involved in the production of kinins, principally bradykinin and Lys-bradykinin (kallidin). The KKS contributes to a variety of physiological processes including inflammation, blood pressure control and coagulation. The activation of this system is particularly important in blood pressure regulation and in inflammatory reactions, due to the ability of bradykinin to elevate vascular permeability and to cause vasodilatation of arteries and veins of the gut, aorta, uterus and urethra. The kinin-kallikrein system, also referred to as the contact system, consists of three serine proenzymes (factor XII (FXII) or Hageman factor, factor IX (FIX), and prekallikrein), and the kinin precursor high molecular weight kinin (HK). Contact activation is triggered by the binding of FXII to a negatively charged surface and involves the formation of α-FXIIa via autocatalysis. Bound α-FXIIa converts prekallikrein into kallikrein. Kallikrein can further convert α-FXIIa to β-FXIIa by an additional cleavage at R334-N335, a positive feedback mechanism that leads to sufficient kallikrein production to drive downstream processes. α-FXIIa consists of a heavy and light chain that are disulphide linked, whereas β-FXIIa lacks the heavy chain and loses its capacity to bind to negatively charged surfaces (Stavrou E, Schmaier A H., Thrombosis Research, 2010, 125(3) pp. 210-215). The N-terminal region of FXII (α-FXIIa heavy chain) shows strong homology with tissue-type plasminogen activator (tPA), with the presence of fibronectin type I, epidermal growth factor, and Kringle domains (Ny et al., Proc Natl Acad Sci USA, 1984, 81(17) pp. 5355-5359; Cool D E, MacGillivray R T, The Journal of Biological Chemistry, 1987, 262(28) pp. 13662-13673). Kallikrein is a trypsin-like serine protease enzyme that cleaves high molecular weight kinin (HK) to produce bradykinin. Bradykinin then binds to the bradykinin 2R receptors (BK2R) on endothelial cells to trigger an increase in vascular permeability.

Protease inhibitors regulate the activation of the contact system. Several known serpins of plasma are C1-inhibitor (C1INH), antithrombin III, α2-macroglobulin, α1-protease inhibitor, and α2-antiplasmin (Kaplan et al., Advances in Immunology, 1997 (66) pp. 225-72; Pixley et al., The Journal of Biological Chemistry, 1985, 260(3) pp. 1723-9). However, C1INH is the major regulator of the intrinsic system, interfering with the activities of factor XIIa and of kallikrein (Cugno et al., The Journal of Laboratory and Clinical Medicine, 1993, 121(1) pp. 38-43). Both C1INH and α2-macroglobulin account for more than 90% of the kallikrein inhibitory activity of plasma. Thus, the FXII-dependent kallikrein-kinin system is tightly regulated by the CINH and when regulation of the FXII-dependent kallikrein-kinin system fails, in a subject, the subject is believed to suffer from hereditary angioedema (HAE) that is characterized by invalidating edema attacks.

Angioedema is a potentially fatal blood disorder characterized by swelling that may occur in the face, gastrointestinal tract, extremities, genitals and upper airways. Angioedema attacks begin in the deeper layers of the skin and mucous membranes with localized blood vessel dilatation and increased permeability. Symptoms of the disease result from the leakage of plasma from blood vessels into surrounding tissues. Genetic hereditary angioedema attacks result from unregulated activation of the kallikrein system with consequent overproduction of bradykinin and uncontrolled increases in vascular permeability. As vascular permeability rises beyond normal, plasma leaks out of the vasculature into surrounding tissue, causing swelling (Mehta D and Malik A B, Physiol. Rev., 86 (1), 279-367, 2006; Sandoval R et al., J. Physiol., 533(pt 2), 433-45, 2001; Kaplan AP and Greaves M W, Angioedema. J. Am. Acad. Dermatol., 2005).

HAE results from mutations in the genes that code for elements of the coagulation and inflammation pathways. The three forms of HAE are distinguished by their underlying causes and levels of the C1-esterase inhibitor (C1INH, serpin peptidase inhibitor, Glade G, member 1) protein in the blood, which inhibits the activity of plasma kallikrein. In type I, patients have insufficient levels of functional C1INH, while type II patients have dysfunctional C1INH. While type I and II affect men and women at equal rates, type III, which primarily affects women, results from a mutation in coagulation factor XII (Hageman factor; HAE-FXII). The underlying causes of type I and II HAE are autosomal dominant mutations in C1INH gene (SERPING1 gene) on chromosome 11 (11q12-q13.1).

C1INH accounts for 90% of inhibition of FXIIa and 50% of inhibition of plasma kallikrein (Pixley R A et al., J. Biol. Chem., 260, 1723-9, 1985; Schapira M et al., Biochemistry, 20, 2738-43, 1981). In addition, C1INH also inactivates prekallikrein (Colman R W et al, Blood, 65, 311-8, 1985). When C1INH levels are normal, its activity blocks FXIIa from converting pre-kallikrein to kallikrein and blocks kallikrein's conversion to HK, thus preventing the production of bradykinin and the edemic episodes. When C1INH levels are low, or levels of dysfunctional C1INH are high, this inhibition fails and the pathogenic process ensues.

In addition to HAE, plasma kallikrein also contributes to non-hereditary angioedema, high altitude cerebral edema, cytotoxic cerebral edema, osmotic cerebral edema, diabetic macular edema (DME), clinically significant macular edema, cystoid macular edema (CME, Gao B B, Nat Med., 13(2), 181-8, 2007), retinal edema, radiation induced edema, lymph edema, glioma-associated edema, allergic edema e.g. airflow obstruction in chronic allergic sinusitis or perennial rhinitis. Other disorders of the plasma kallikrein system include retinopathy and diabetic retinopathy (Liu J and Feener E P, Biol. Chem. 394(3), 319-28, 2013), proliferative and non-proliferative retinopathy (Liu J et al, Invest. Ophthalmol. Vis. Sci., 54(2), 2013), CME following cataract extraction, CME induced by cryotherapy, CME induced by uveitis, CME following vascular occlusion (e.g., central retinal vein occlusion, branch retinal vein occlusion or hemiretinal vein occlusion), complications related to cataract surgery in diabetic retinopathy, hypertensive retinopathy (J A Phillips et al., Hypertension, 53, 175-181, 2009), retinal trauma, dry and wet age-related macular degeneration (AMD), ischemic reperfusion injuries (C Storoni et al., JPET, 381, 849-954, 2006), e.g., in a variety of contexts associated with tissue and/or organ transplantation.

Current treatments for angioedema, and those under development, target different elements in the HAE pathway. Three classes of therapies are currently available: (a) replacement therapy with C1INH concentrates (e.g., Cinryze, Berinert), (b) administration of selective kallikrein inhibitors (e.g., Ecallantide) and (c) bradykinin receptors antagonists (e.g., Firazyr).

Replacement therapies have proven useful for both acute attacks, including emergency situations, such as laryngeal edema (Bork K et al., Transfusion, 45, 1774-1784, 2005; Bork K and Barnstedt S E, Arch. Intern. Med., 161, 714-718, 2001) and prophylaxis. Selective C1INH inhibitors inactivate both α-FXIIa and β-FXIIa molecules active early in the HAE pathway that catalyze the production of kallikrein (Muller F and Renne T, Curr. Opin. Hematol., 15, 516-21, 2008; Cugno M et al., Trends Mol. Med. 15(2):69-78, 2009). In addition to HAE, plasma kallikrein inhibitors are considered to be useful in the treatment of other edemas such as macular edema and brain edema, and retinopathy, e.g., retinopathy associated with diabetes and/or hypertension. There is evidence that plasma kallikrein inhibitors are also effective in the treatment of edema formation in diseases, e.g., edema formation related to ischemic reperfusion injuries. The bradykinin receptors antagonists prevent bradykinin from activating the vascular permeability pathway and stop the initiation of swelling.

### Kallikrein Inhibitor

Provided herein is the kallikrein inhibitor N-((6-amino-2,4-dimethylpyridin-3-yl)methyl)-2-((3-chloroquinolin-6-yl)methyl)isonicotinamide, also known as ATN-249 (also referred to herein as compound A). Compound A has been disclosed in WO 2016/011209 and in WO 2015/103317. The structure of Compound A is provided below.

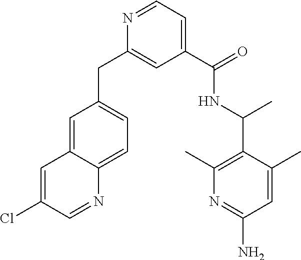 [SMILES: Cc1cc(N)nc(C)c1C(C)NC(=O)c1ccnc(Cc2ccc3ncc(Cl)cc3c2)c1]

One embodiment provides a method of inhibiting kallikrein enzyme comprising contacting the enzyme with N-((6-amino-2,4-dimethylpyridin-3-yl)methyl)-2-((3-chloroquinolin-6-yl)methyl)isonicotinamide.

One embodiment provides a method of inhibiting plasma kallikrein in a subject comprising administering to the subject a composition comprising N-((6-amino-2,4-dimethylpyridin-3-yl)methyl)-2-((3-chloroquinolin-6-yl)methyl)isonicotinamide, or a pharmaceutically acceptable salt thereof.

### Methods of Treatment

Disclosed herein are methods of treating diseases or disorders wherein the inhibition of plasma kallikrein is indicated. Such diseases and disorders include but are not limited to angioedema, including hereditary and non-hereditary.

In some embodiments, the methods disclosed herein are useful for the treatment of angioedema. In some embodiments, the angioedema is hereditary angioedema (HAE). One embodiment provides a method of treating angioedema in a patient in need thereof comprising admisitration of a composition comprising a N-((6-amino-2,4-dimethylpyridin-3-yl)methyl)-2-((3-chloroquinolin-6-yl)methyl)isonicotinamide, or a pharmaceutically acceptable salt thereof. Another embodiment provides the method wherein the angioedema is hereditary angioedema.

One embodiment provides N-((6-amino-2,4-dimethylpyridin-3-yl)methyl)-2-((3-chloroquinolin-6-yl)methyl)isonicotinamide, or a pharmaceutically acceptable salt thereof, for use in a method of treatment of the human or animal body.

One embodiment provides N-((6-amino-2,4-dimethylpyridin-3-yl)methyl)-2-((3-chloroquinolin-6-yl)methyl)isonicotinamide, or a pharmaceutically acceptable salt thereof, for use in a method of treatment of angioedema. Another embodiment provides a compound for use wherein the angioedema is hereditary angioedema.

One embodiment provides the use of N-((6-amino-2,4-dimethylpyridin-3-yl)methyl)-2-((3-chloroquinolin-6-yl)methyl)isonicotinamide, or a pharmaceutically acceptable salt thereof, in the manufacture of a medicament for the treatment of angioedema. Another embodiment provides the use wherein the angioedema is hereditary angioedema.

One embodiment provides a method of treating angioedema in a patient in need thereof comprising administering a composition comprising N-((6-amino-2,4-dimethylpyridin-3-yl)methyl)-2-((3-chloroquinolin-6-yl)methyl)isonicotinamide, or a pharmaceutically acceptable salt thereof. Another embodiment provides the method wherein the angioedema is hereditary angioedema.

### Pharmaceutical Compositions

In certain embodiments, N-((6-amino-2,4-dimethylpyridin-3-yl)methyl)-2-((3-chloroquinolin-6-yl)methyl)isonicotinamide is administered as a pure chemical. In other embodiments, N-((6-amino-2,4-dimethylpyridin-3-yl)methyl)-2-((3-chloroquinolin-6-yl)methyl)isonicotinamide is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Provided herein is a pharmaceutical composition comprising N-((6-amino-2,4-dimethylpyridin-3-yl)methyl)-2-((3-chloroquinolin-6-yl)methyl)isonicotinamide, or a stereoisomer, pharmaceutically acceptable salt, hydrate, solvate, or N-oxide thereof, together with one or more pharmaceutically acceptable carriers. The carrier(s) (or excipient(s)) is acceptable or suitable if the carrier is compatible with the other ingredients of the composition and not deleterious to the recipient (i.e., the subject) of the composition.

One embodiment provides a pharmaceutical composition comprising N-((6-amino-2,4-dimethylpyridin-3-yl)methyl)-2-((3-chloroquinolin-6-yl)methyl)isonicotinamide, or a pharmaceutically acceptable salt thereof, and a pharmaceutically acceptable excipient.

In certain embodiments, N-((6-amino-2,4-dimethylpyridin-3-yl)methyl)-2-((3-chloroquinolin-6-yl)methyl)isonicotinamide, or a pharmaceutically acceptable salt thereof, is substantially pure, in that it contains less than about 5%, or less than about 1%, or less than about 0.1%, of other organic small molecules, such as unreacted intermediates or synthesis by-products that are created, for example, in one or more of the steps of a synthesis method.

Suitable oral dosage forms include, for example, tablets, pills, sachets, or capsules of hard or soft gelatin, methylcellulose or of another suitable material easily dissolved in the digestive tract. In some embodiments, suitable nontoxic solid carriers are used which include, for example, pharmaceutical grades of mannitol, lactose, starch, magnesium stearate, sodium saccharin, talcum, cellulose, glucose, sucrose, magnesium carbonate, and the like. (See, e.g., Remington: The Science and Practice of Pharmacy (Gennaro, 21^{st} Ed. Mack Pub. Co., Easton, Pa. (2005)).

The dose of the composition comprising N-((6-amino-2,4-dimethylpyridin-3-yl)methyl)-2-((3-chloroquinolin-6-yl)methyl)isonicotinamide differ, depending upon the patient's (e.g., human) condition, that is, stage of the disease, general health status, age, and other factors.

Pharmaceutical compositions are administered in a manner appropriate to the disease to be treated (or prevented). An appropriate dose and a suitable duration and frequency of administration will be determined by such factors as the condition of the patient, the type and severity of the patient's disease, the particular form of the active ingredient, and the method of administration. In general, an appropriate dose and treatment regimen provides the composition(s) in an amount sufficient to provide therapeutic and/or prophylactic benefit (e.g., an improved clinical outcome, such as more frequent complete or partial remissions, or longer disease-free and/or overall survival, or a lessening of symptom severity. Optimal doses are generally determined using experimental models and/or clinical trials. The optimal dose depends upon the body mass, weight, or blood volume of the patient.

Oral doses typically range from about 1.0 mg to about 1000 mg, one to four times, or more, per day.

One embodiment provides a pharmaceutical composition comprising N-((6-amino-2,4-dimethylpyridin-3-yl)methyl)-2-((3-chloroquinolin-6-yl)methyl)isonicotinamide, or a pharmaceutically acceptable salt thereof, and a pharmaceutically acceptable excipient.

One embodiment provides a method of preparing a pharmaceutical composition comprising mixing N-((6-amino-2,4-dimethylpyridin-3-yl)methyl)-2-((3-chloroquinolin-6-yl)methyl)isonicotinamide, or a pharmaceutically acceptable salt thereof, and a pharmaceutically acceptable carrier.

In an aspect, provided herein is N-((6-amino-2,4-dimethylpyridin-3-yl)methyl)-2-((3-chloroquinolin-6-yl)methyl)isonicotinamide, or a pharmaceutically acceptable salt thereof, for use in a method of treatment of the human or animal body.

In another aspect, provided herein is a method of treating angioedema in a patient in need thereof, comprising admisitration of a composition comprising N-((6-amino-2,4-dimethylpyridin-3-yl)methyl)-2-((3-chloroquinolin-6-yl)methyl)isonicotinamide, or a pharmaceutically acceptable salt thereof.

In some embodiments, the angioedema is hereditary angioedema.

In some embodiments, the composition is administered daily. In some embodiments, the composition is administered once or twice per day. In some embodiments, the N-((6-amino-2,4-dimethylpyridin-3-yl)methyl)-2-((3-chloroquinolin-6-yl)methyl)isonicotinamide is administered twice per day. In some embodiments, the N-((6-amino-2,4-dimethylpyridin-3-yl)methyl)-2-((3-chloroquinolin-6-yl)methyl)isonicotinamide is administered once per day.

In some embodiments, the composition is administered orally.

In some embodiments, the N-((6-amino-2,4-dimethylpyridin-3-yl)methyl)-2-((3-chloroquinolin-6-yl)methyl)isonicotinamide is administered in an amount of from about 100 mg/day to about 800 mg/day. In some embodiments, the N-((6-amino-2,4-dimethylpyridin-3-yl)methyl)-2-((3-chloroquinolin-6-yl)methyl)isonicotinamide is administered in an amount of from about 300 mg/day to about 800 mg/day.

In some embodiments, the N-((6-amino-2,4-dimethylpyridin-3-yl)methyl)-2-((3-chloroquinolin-6-yl)methyl)isonicotinamide is administered in an amount of about 100 mg/day, about 150 mg/day, about 200 mg/day, about 250 mg/day, about 300 mg/day, about 350 mg/day, about 400 mg/day, about 450 mg/day, about 500 mg/day, about 600 mg/day, about 650 mg/day, about 700 mg/day, about 750 mg/day, or about 800 mg/day.

In some embodiments, the N-((6-amino-2,4-dimethylpyridin-3-yl)methyl)-2-((3-chloroquinolin-6-yl)methyl)isonicotinamide is administered in an amount of about 300, about 350 mg/day, about 400 mg/day, about 450 mg/day, about 500 mg/day, about 600 mg/day, about 650 mg/day, about 700 mg/day, about 750 mg/day, or about 800 mg/day. In some embodiments, the N-((6-amino-2,4-dimethylpyridin-3-yl)methyl)-2-((3-chloroquinolin-6-yl)methyl)isonicotinamide is administered in an amount of about 400 mg/day, about 450 mg/day, about 500 mg/day, about 600 mg/day, about 650 mg/day, about 700 mg/day, about 750 mg/day, or about 800 mg/day. In some embodiments, the N-((6-amino-2,4-dimethylpyridin-3-yl)methyl)-2-((3-chloroquinolin-6-yl)methyl)isonicotinamide is administered in an amount of about 400 mg/day. In some embodiments, the N-((6-amino-2,4-dimethylpyridin-3-yl)methyl)-2-((3-chloroquinolin-6-yl)methyl)isonicotinamide is administered in an amount of about 450 mg/day. In some embodiments, the N-((6-amino-2,4-dimethylpyridin-3-yl)methyl)-2-((3-chloroquinolin-6-yl)methyl)isonicotinamide is administered in an amount of about 500 mg/day. In some embodiments, the N-((6-amino-2,4-dimethylpyridin-3-yl)methyl)-2-((3-chloroquinolin-6-yl)methyl)isonicotinamide is administered in an amount of about 550 mg/day. In some embodiments, the N-((6-amino-2,4-dimethylpyridin-3-yl)methyl)-2-((3-chloroquinolin-6-yl)methyl)isonicotinamide is administered in an amount of about 600 mg/day. In some embodiments, the N-((6-amino-2,4-dimethylpyridin-3-yl)methyl)-2-((3-chloroquinolin-6-yl)methyl)isonicotinamide is administered in an amount of about 650 mg/day. In some embodiments, the N-((6-amino-2,4-dimethylpyridin-3-yl)methyl)-2-((3-chloroquinolin-6-yl)methyl)isonicotinamide is administered in an amount of about 700 mg/day. In some embodiments, the N-((6-amino-2,4-dimethylpyridin-3-yl)methyl)-2-((3-chloroquinolin-6-yl)methyl)isonicotinamide is administered in an amount of about 750 mg/day. In some embodiments, the N-((6-amino-2,4-dimethylpyridin-3-yl)methyl)-2-((3-chloroquinolin-6-yl)methyl)isonicotinamide is administered in an amount of about 800 mg/day.

In some embodiments, the N-((6-amino-2,4-dimethylpyridin-3-yl)methyl)-2-((3-chloroquinolin-6-yl)methyl)isonicotinamide is administered in an amount of about 400 mg/day, about 450 mg/day, about 500 mg/day, about 600 mg/day, about 650 mg/day, about 700 mg/day, about 750 mg/day, or about 800 mg/day. In some embodiments, the N-((6-amino-2,4-dimethylpyridin-3-yl)methyl)-2-((3-chloroquinolin-6-yl)methyl)isonicotinamide is administered in an amount of about 400 mg/day.

In some embodiments, the N-((6-amino-2,4-dimethylpyridin-3-yl)methyl)-2-((3-chloroquinolin-6-yl)methyl)isonicotinamide is administered in an amount of about 300 mg. In some embodiments, the N-((6-amino-2,4-dimethylpyridin-3-yl)methyl)-2-((3-chloroquinolin-6-yl)methyl)isonicotinamide is administered in an amount of about 300 mg twice per day.

In some embodiments, the composition is formulated for immediate release.

In some embodiments, the composition is formulated for as a tablet or capsule.

In some embodiments, the composition further comprises at least one pharmaceutically acceptable excipient.

Other embodiments and uses will be apparent to one skilled in the art in light of the present disclosures. The following examples are provided merely as illustrative of various embodiments and shall not be construed to limit the invention in any way.

### EXAMPLES

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed in practicing the invention. It is intended that the following claims define the scope of the invention and that methods and structures within the scope of these claims and their equivalents be covered thereby.

### Example 1

### Selectivity, Potency, and Exposure Evaluation of N-((6-amino-2,4-dimethylpyridin-3-yl)methyl)-2-((3-chloroquinolin-6-yl)methyl)isonicotinamide, A New Oral Kallikrein Inhibitor for Hereditary Angioedema

Hereditary angioedema (HAE) is a rare, potentially life threatening disease characterized by acute skin and mucosal edema. HAE may result in recurrent skin swelling, abdominal pain, laryngeal edema, nonerythematous rash, tingling sensations, anxiety, mood changes, or exhaustion. HAE is caused by a deficiency of C1 inhibitor (C1-INH), which leads to increased levels of plasma kallikrein. Increased levels of plasma kallikrein lead to elevated levels of bradykinin, which causes vasodilation, inflammation, and edema. Currently, there is an unmet need for orally-administered therapies that control plasma kallikrein activity, prevent HAE attacks, and are well-tolerated

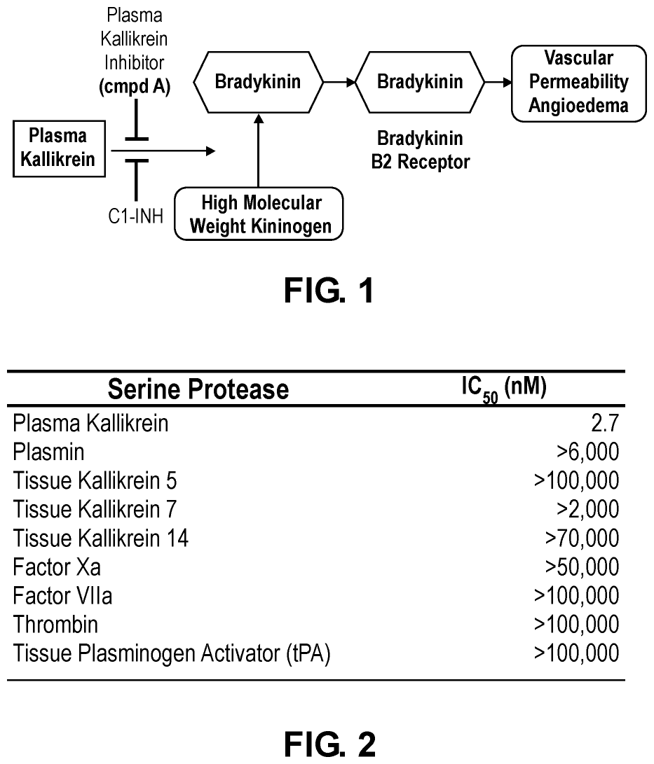

Compound A is a novel, orally-administered plasma kallikrein inhibitor that potentially treats HAE by blocking kallikrein mediated production of bradykinin (FIG. 1).

### Objectives

The objective of this preclinical study was to evaluate the selectivity of Compound A, as well as the potency, pharmacokinetic exposure, and safety of Compound A as compared to C1 inhibitor (C1-INH).

### Materials and Methods

Selectivity was evaluated by biochemical inhibition on plasma kallikrein relative to other serine proteases, including tissue kallikrein 5, plasmin, Factor Xa, Factor VIIa, thrombin, and tissue plasminogen activator (tPA). Potency was evaluated by biochemical inhibition and contact activation assays in human plasma. Pharmacokinetic exposure was evaluated in monkeys after a single oral administration of Compound A at 15 mg/kg. The no-observed-adverse-effect-level (NOAEL) was evaluated in 14-day non-GLP rat and monkey toxicology studies; animals were given daily doses of 100 or 300 mg/kg.

### Results

Compound A was >2000-fold more selective at inhibiting plasma kallikrein versus other closely related serine proteases, including tissue kallikrein 5, plasmin, Factor Xa, Factor VIIa, thrombin, and tissue plasminogen activator (tPA) (FIG. 2).

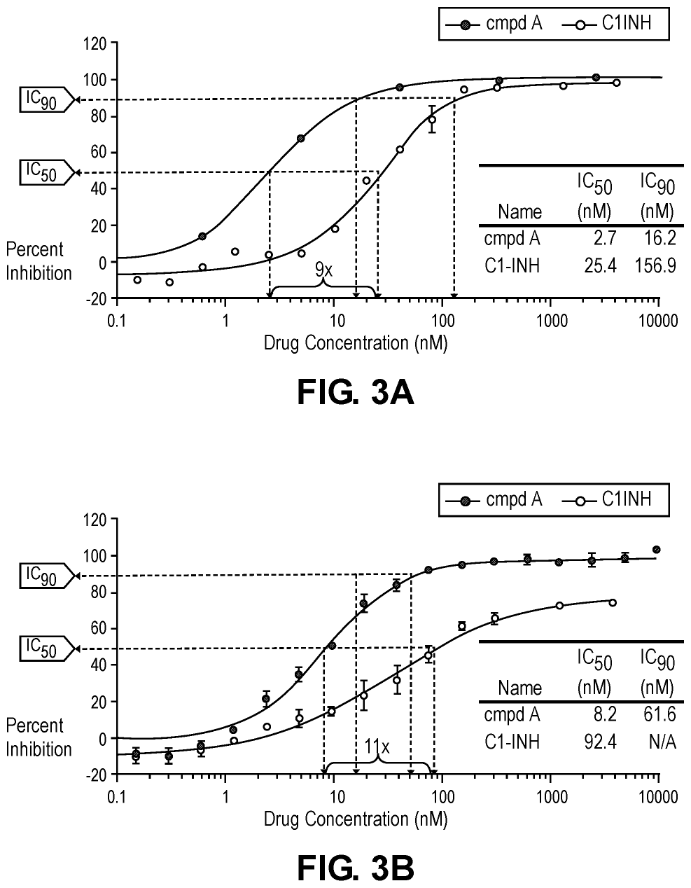

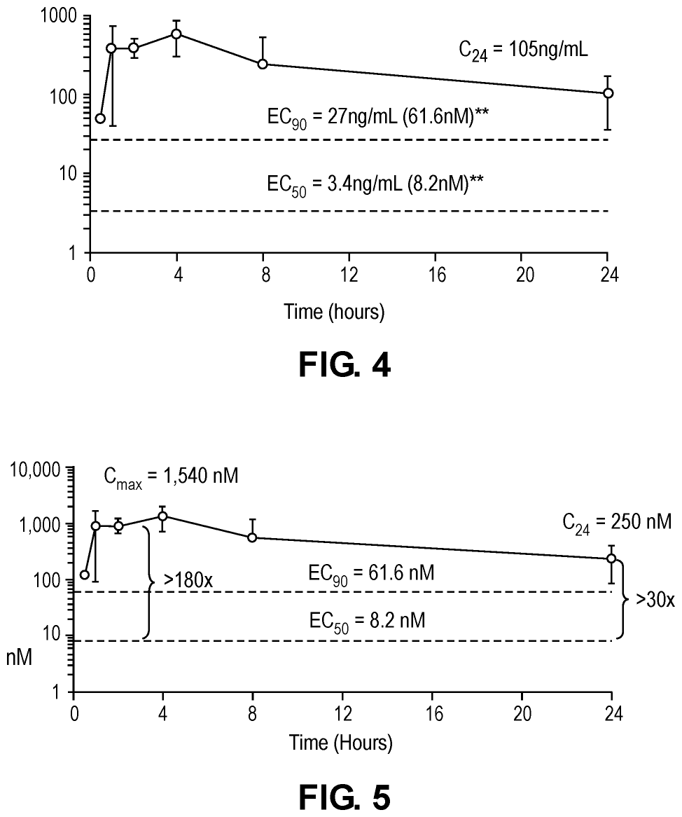

Compound A was 9- to 11-fold more potent than C1-INH at inhibiting plasma kallikrein in both biochemical inhibition and contact activation assays (an ex vivo assay that closely represents clinical pharmacology. In biochemical inhibition, Compound A had an IC₅₀ of 2.7 nM vs 25.4 nM for C1-INH (FIG. 3A); in contact activation assays, the EC₅₀ was 8.2 nM vs 92.4 nM, respectively (FIG. 3B). A pharmacokinetic exposure study showed that a single oral dose of Compound A at 15 mg/kg provided 24-hour exposure (C₂₄) 30-fold greater than EC₅₀ in monkeys (FIG. 4). No adverse events were observed at the highest dose (300 mg/kg), setting the no-observed-adverse-effect-level (NOAEL) at 300 mg/kg.

Compound A was highly selective at plasma kallikrein inhibition compared to other closely related serine proteases. Compound A demonstrated ˜10-fold greater plasma kallikrein inhibition relative to C1-INH in both biochemical inhibition and contact activation assays—an ex vivo assay that closely represents clinical pharmacology. After a single dose, Compound A at 15 mg/kg provided 24-hour exposure 30-fold greater than EC₅₀ and 20-fold below the no-observed-adverse-effect-level (NOAEL). These results suggest a wide therapeutic window and once-daily dosing potential. Compound A may be a potent, safe, orally-administered plasma kallikrein inhibitor for treatment of HAE.

### Example 2

### Preclinical Safety Study of Compound A

There is a strong unmet need for effective, well-tolerated, safe oral therapies with improved patient quality of life, convenience and prophylactic efficacy. Acute therapies and prophylactic I.V. and S.C. therapies for treating HAE are also desirable.

### Main Objectives:

Compound A was selected for the study on the basis of chemical structure, selectivity for plasma kallikrein, and kallikrein inhibition.

The objectives of the study were as follows:
(1) Evaluate the potency of Compound A compared to C1-INH via inhibition of plasma kallikrein;
(2) Evaluate the selectivity of Compound A on biochemical inhibition of plasma kallikrein relative to other closely related serine proteases; and
(3) Evaluate the general toxicity, safety pharmacology, and genotoxicity profiles of Compound A.

### Potency:

Compound A was 9-fold more potent than C1-INH at inhibiting plasma kallikrein in a biochemical inhibition assay (FIG. 3A). Compound A was 11-fold more potent than C1-INH at inhibiting plasma kallikrein in a contact activation assay (FIG. 3B).

### Selectivity:

Compound A was >2000-fold more selective at inhibiting plasma kallikrein versus other closely related serine proteases (FIG. 2).

### Safety:

A pharmacokinetic exposure study showed that a single oral dose of Compound A at 15 mg/kg provided Cₘₐₓ exposure>180× and 24 hour exposure (C₂₄) 30-fold>EC₅₀ in monkeys (FIG. 5).

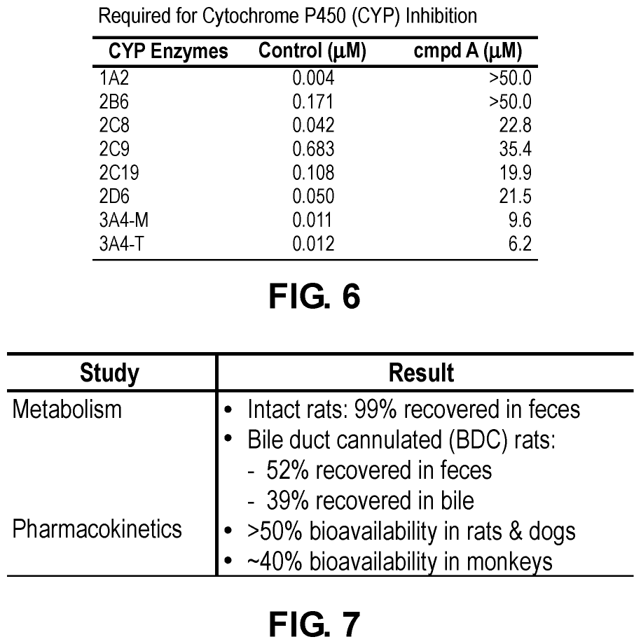

Compound A was found to not significantly inhibit P450 enzymes (FIG. 6).

In metabolism and pharmacokinetics studies of single oral administration/dosing, Compound A demonstrated good bioavailability in all species and comprehensive recovery of radiolabeled Compound A (FIG. 7).

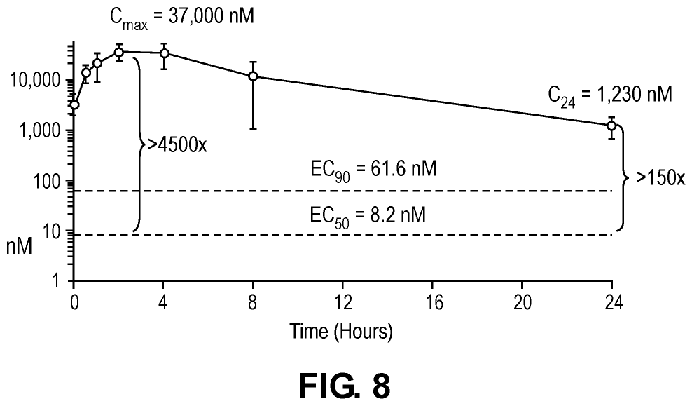

General toxicity Compound A 28-day repeat dose studies were conducted in rats and monkeys (FIG. 8). At no-observed-adverse-effect-level (NOAEL) dose, Compound A provided Cₘₐₓ exposure>4500× and 24-h exposure (C₂₄) 150×>EC₅₀ at day 28.

In rats, NOAEL of 300 mg/kg/day, high-dose level resulted in decreases in body weight and food consumption—the 300 mg/kg/day level was not considered adverse. In monkeys, NOAEL was 100 mg/kg/day, mid-dose level. 300 mg/kg/day high-dose level adverse findings in monkeys reversed upon dose reduction to 150 mg/kg/day dose.

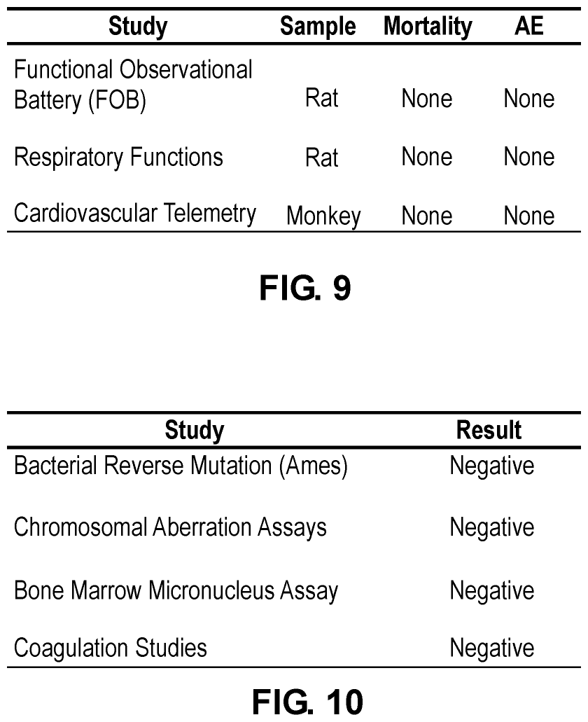

In Compound A single dose rat and monkey safety pharmacology studies, no mortality or adverse effects were observed on central nervous system, respiratory, and cardiovascular functions (FIG. 9). In Compound A toxicology and genotoxicity studies, no genotoxicity or coagulation issues were noted in a wide range of studies (FIG. 10).

The results from the safety studies evaluating Compound A, an orally administered plasma kallikrein inhibitor for the treatment of Hereditary Angioedema (HAE), were positive. The strong safety, high potency, and high selectivity results suggest a wide therapeutic window with once-daily dosing potential of Compound A. In the preclinical toxicology and safety pharmacology studies, Compound A was generally safe and well tolerated. In addition, pharmacokinetic studies indicated high 24-hour exposure and comprehensive drug recovery after repeat oral doses of Compound A. This data indicates that Compound A, has a favorable safety profile and once-a day dosing regimen to address the unmet need for well-tolerated and safe oral therapies with improved patient quality life and prophylactic efficacy.

Studies included evaluation of potency of Compound A compared to C1-INH via inhibition of plasma kallikrein, selectivity of Compound A on biochemical inhibition of plasma kallikrein relative to other closely related serine proteases, and Compound A's pharmacokinetics, general toxicity, safety pharmacology, and genotoxicity profiles.

Safety:

No-observed-adverse-effect-level (NOAEL) was established at 100 mg/kg/day, mid-dose level in monkeys.

No mortality or adverse effects were observed on central nervous system, respiratory, and cardiovascular functions in safety pharmacology studies.

No genotoxicity or coagulation issues were noted in a wide range of studies.

DMPK:

High 24-hour exposure, comprehensive drug recovery, no P450 liabilities.

After repeat doses at the NOAEL dose of 100 mg/kg/day, Compound A provided Cₘₐₓ exposure>600-fold and 24-h exposure 20-fold higher than EC₉₀ at day 28.

After single oral administration of 30 mg/kg, Compound A demonstrated>40% bioavailability in rats, dogs, and monkeys.

After single oral administration of 15 mg/kg in monkeys, Compound A provided Cₘₐₓ exposure 25-fold and 24-h exposure 4-fold higher than EC₉₀ Compound A demonstrated 99% recovery in intact and bile duct cannulated rats after single oral dosing.

Compound A does not significantly inhibit P450 enzymes.

Potency:

Compound A demonstrated ˜10-fold greater plasma kallikrein inhibition relative to C1-INH in both biochemical inhibition and contact activation assays—an ex vivo assay that closely represents clinical pharmacology.

In biochemical inhibition, Compound A had an IC₅₀ of 2.7 nM and an IC₉₀ of 16.2nM versus 25.4 nM and 156.9 nM, respectively for C1-INH.

In contact activation assays, Compound A had an EC₅₀ and EC₉₀ of 8.2 nM and 61.6 nM versus 92.4 nM and N/A, respectively for C1-INH.

### Selectivity:

Compound A was >2000-fold more selective at inhibiting plasma kallikrein versus other closely related serine proteases, including tissue kallikrein 5, tissue kallikrein 7, tissue kallikrein 14, plasmin, Factor Xa, Factor VIIa, thrombin, and tissue plasminogen activator (tPA)

Studies in both biochemical and contact activation assays have demonstrated that Compound A is highly selective and potent at plasma kallikrein inhibition. Compound A has been evaluated in several pharmacokinetic and toxicological studies in multiple species. Given its observed wide therapeutic window and once-daily dosing potential, these results suggest that Compound A may be a potent, safe, orally-administered plasma kallikrein inhibitor for the treatment of HAE.

### Example 3

### Safety, Tolerability, Pharmacokinetics and Food Effect of N-((6-amino-2,4-dimethylpyridin-3-yl)methyl)-2-((3-chloroquinolin-6-yl)methyl)isonicotinamide in Healthy Volunteers

A randomized, double-blind, placebo-controlled, single-ascending-dose and two-way crossover food effect study to determine the safety, tolerability, pharmacokinetics and food effect of Compound A in healthy male participants.

The primary aims of this first-in-human study are to investigate the safety and tolerability of Compound A, and pharmacokinetics when fasting and following high fat meal. The secondary aim is to investigate the pharmacodynamics of Compound A related to contact pathway activation. Up to 24 participants will be recruited to three Cohorts of 8 participants each in this double-blind study.

Participants in Cohort 1 will be randomized to receive an oral dose of either 50 mg (1×50 mg capsule) of Compound A (6 participants) or placebo (2 participants). Two sentinel participants (one allocated to placebo and one allocated to Compound A) will be dosed initially. If dosing of these sentinel participants proceeds without clinically-significant adverse events (AEs) over 24 hours (as adjudicated by the SMC), the remaining 6 participants will be dosed. Participants will be dosed following an overnight fasting of at least 10 hours.

Cohort 2 will be subject to a crossover design with two treatment periods. Participants will be randomized to receive either 100 mg (2×50 mg capsules) of Compound A (6 participants) or placebo (2 participants). Dosing will follow at least 10 hours overnight fasting in the first treatment period; and high fat meal in the second treatment period. The wash-out period between treatments will be of at least 7 days. As with Cohort 1, two sentinel participants (one allocated to placebo and one allocated to Compound A) will be dosed initially during the first treatment period. The planned study procedures for Cohort 2 will proceed if dosing of these sentinel participants proceeds without clinically significant AEs.

Cohort 3 will be analogous to Cohort 1 in terms of study procedures. The dose level will be established following assessment of safety and PK data of the preceding cohorts.

Primary Outcome Measures:
(1): Safety and tolerability of Compound A; Timepoint (1): Up to 7 days following last administration
(2): Plasma concentration and pharmacokinetic parameters of Compound A in fasted state, Timepoint (2): Up to 48 hours following last administration
(3): Plasma concentration and pharmacokinetic parameters of Compound A following ingestion of high fat meal; Timepoint (3): Up to 48 hours following last administration

Secondary Outcome Measure: Pharmacodynamics of Compound A on contact pathway activation; Timepoint: Up to 24 hours following last administration

Key Inclusion Criteria:
1) Male healthy volunteers, age 18 to 55 years, inclusive;
2) Participants must be in good general health, with no significant medical history, have no clinically significant abnormalities on physical examination at screening, and/or before administration of the initial dose of study drug;
3) Participants must have a Body Mass Index (BMI) between 18.0 and 30.0 kg/m² inclusive;
4) Participants must have clinical laboratory values within normal range as specified by the testing laboratory, unless deemed not clinically significant by the Investigator or delegate;
5) Participants must be a non-smoker, and must not have used any tobacco products within six months prior to screening;
6) Participant must have no relevant dietary restrictions, and be willing to consume standard meals provided;
7) Participants who have not been sterilized must make a commitment to ensure that their partners (if of child bearing potential) use highly effective contraception during the period from dosing to 7 days postdose (acceptable forms of contraception are oral, injected or implanted hormonal methods, or placement of an intrauterine device or intrauterine system, or abstinence); in addition to these measures, male participants should use a condom for sexual intercourse during this period. This requirement does not apply to participants in same sex relationships;
8) Participants must have the ability and willingness to attend the necessary visits to the study center;
9) Written informed consent signed prior to entry into the study.

Key Exclusion Criteria:
1) Prior or ongoing medical condition, medical history, physical findings, or laboratory abnormality that, in the Investigator's (or delegate's) opinion, could adversely affect the safety of the participant.
2) Mentally or legally incapacitated, has significant emotional problems at the time of screening visit or expected during the conduct of the study, or has a history of a clinically significant psychiatric disorder within the last 5 years. Note: Participants who have had situational depression may be enrolled in the study at the discretion of the investigator or delegate.
3) Fever (body temperature>38 ° C.) or symptomatic viral or bacterial infection within 2 weeks prior to screening.
4) History of severe allergic or anaphylactic reactions.
5) Resting blood pressure>140/90 mm Hg, resting heart rate>90 beats per minute or resting heart rate<50 beats per minute at screening or at Day −1 (repeat measurements are allowed at the discretion of the Investigator, except for resting heart rate<50 beats per minute).
6) Alkaline phosphatase (ALP), aspartate aminotransferase (AST) and/or alanine aminotransferase (ALT)>1.5×upper limit of normal at screening. Repeat testing at screening is acceptable for out of range values following approval by the Investigator or delegate.
7) Serum potassium<3.7 mmol/L or >5.5 mmol/L at Screening or Day −1.
8) Positive test for hepatitis C antibody, hepatitis B surface antigen, or human immunodeficiency virus (HIV) antibody at screening.
9) Participants with a positive toxicology screening panel (urine test including qualitative identification of barbiturates, Tetrahydrocannabinol (THC), amphetamines, benzodiazepines, opiates and cocaine).
10) Participants with a history of substance abuse or dependency or history of recreational intravenous (IV) drug use over the last years (by self-declaration).
11) Regular alcohol consumption defined as >21 alcohol units per week (where 1 unit=284 mL of beer, 25 ml of 40% spirit or a 125 ml glass of wine). Participant is unwilling to abstain from alcohol beginning 48 hours prior to admission to the CRU until follow-up visit.
12) Participant has significant ECG abnormalities that might interfere with ECG analysis including evidence of a previous myocardial infarction (MI), left ventricular hypertrophy (LVH), flat T waves (particularly in the inferior leads) or more than minor non-specific ST-T wave changes or:
   a. QRS>110 milliseconds (msec),
   b. QT interval corrected using Fridericia's formula (QTcF)>440 msec (men and women),
   c. PR interval>220 msec
   d. Heart rate<50 BPM or >90 BPM
   e. Complete right bundle branch block or left bundle branch block.
13) History of cardiac disease or cerebrovascular disease, including coronary artery disease (including MI, angina), cardiac arrhythmias, long QT syndrome (in self or family), valvular disease, heart failure, hypertension or hypotension.
14) Family history of hereditary angioedema.
15) Use of any prescription medication, over-the-counter medication, herbal products, vitamins or minerals, within 7 days or 5 half-lives (whichever is longer) prior to study drug administration, unless in the opinion of the Principal Investigator and/or Medical Monitor the medication will not compromise participant safety or interfere with study procedures or data validity.
16) Use of any potential inducer or inhibitor of cytochrome P450 [CYP] 3A4 or P-glycoprotein [P gp] [e.g., St. John's Wort, rifampin, cyclosporine, or ritonavir]) within 14 days or 5 half lives (whichever is longer) prior to study drug administration, unless in the opinion of the Principal Investigator and/or Medical Monitor the medication will not compromise participant safety or interfere with study procedures or data validity.
17) Anticipated use of prescription medication or over-the-counter medication during study participation, with the exception of 1-2 therapeutic doses per week of paracetamol/acetaminophen or non-steroidal anti-inflammatory drugs (e.g., ibuprofen, naproxen).
18) Participant is unwilling to refrain from strenuous exercise from 7 days prior to admission to the CRU through discharge from the entire study
19) Participant is unwilling to abstain from ingestion of caffeine or xanthine-containing products (e.g., tea, coffee, chocolate, cola, etc.) beginning 96 hours prior to admission to the CRU for each study period until the final pharmacokinetic (PK) sample of each study period has been collected.
20) Participant has consumed grapefruit and/or grapefruit juice within 14 days prior to admission to the CRU and is unwilling to abstain from consuming grapefruit and/or grapefruit juice until the end of the study.
21) Participant has consumed other fruit or fruit juices within 48 hours prior to admission to the CRU for each study period and is unwilling to abstain from these items for 48 hours prior to admission for each study period until the final PK sample of each study period has been collected.
22) Participants who are unlikely to comply with the study protocol or, in the opinion of the investigator, would not be a suitable candidate for participation in the study.

Methods used to generate the sequence in which subjects will be randomized (sequence generation): Simple randomization using a randomization table created using SAS EG 7.12 software package

Individuals receiving the treatment(s); individuals administering the treatment(s); and individuals assessing the outcomes will be blinded/masked.

Other design features: Cohort 1 and 3 follow parallel design; Cohort 2 follows crossover design.

Safety Analysis Set: All participants who received any amount of study drug. PK Analysis Set: All participants who received study drug (Compound A) and have sufficient PK data for analysis.

### Example 4

### Pharmacokinetics and Safety of Compound A, a Novel Oral Plasma Kallikrein Inhibitor for Hereditary Angioedema

Objectives: Assess the safety, tolerability, and pharmacokinetics (PK) (including food effect) of Compound A in healthy male participants in a single-ascending-dose (SAD) study

Materials and Methods

A randomized, double-blind, placebo-controlled single ascending dose and crossover food effect study

48 healthy male participants (6 active:2 placebo in each of the 6 dose cohorts) received a single daily dose of Compound A 50 mg, 100 mg, 150 mg, 200 mg, 400 mg, or 800 mg. Subjects in the 100 mg dose cohort received first dose of Compound A under fasted condition in period 1 and after a 7-day washout, a second dose 30 minutes after the start of a high fat, high caloric meal in period 2. Serial blood draws were collected to calculate PK parameters, including area under the curve (AUC) from time zero to infinity (AUC), maximum concentration (Cₘₐₓ), time of maximum concentration (Tₘₐₓ), and half-life. Safety measures including treatment-emergent adverse events (TEAEs) were assessed.

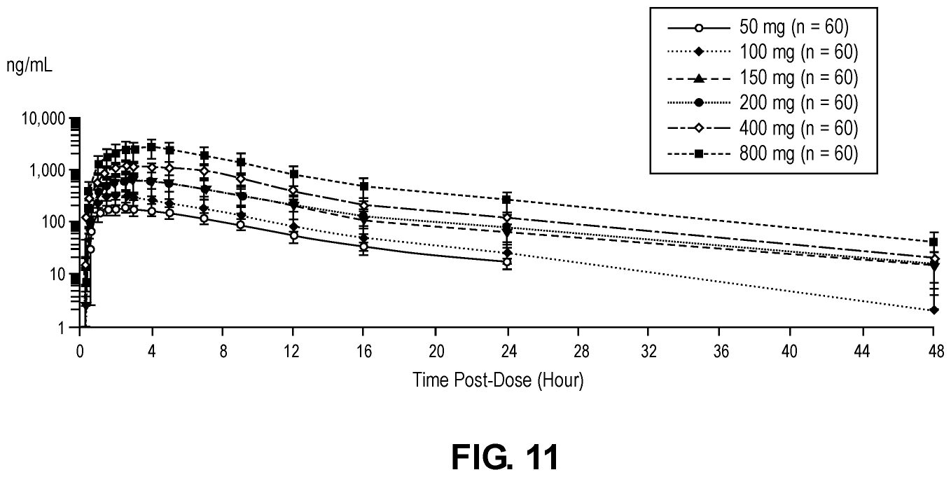

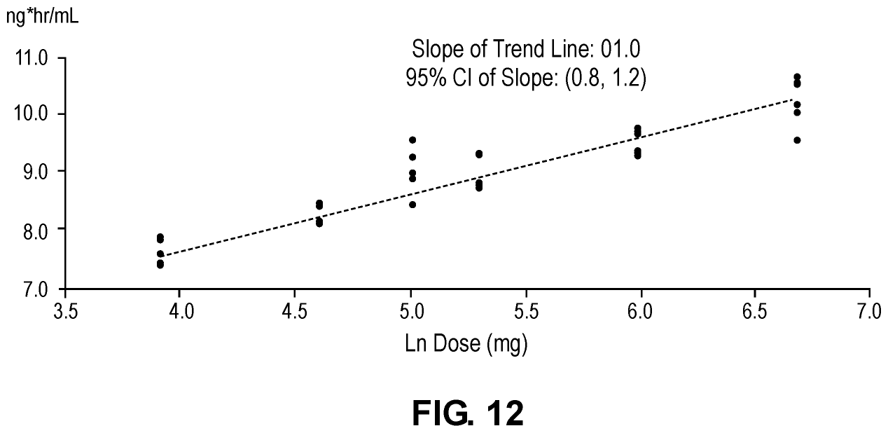

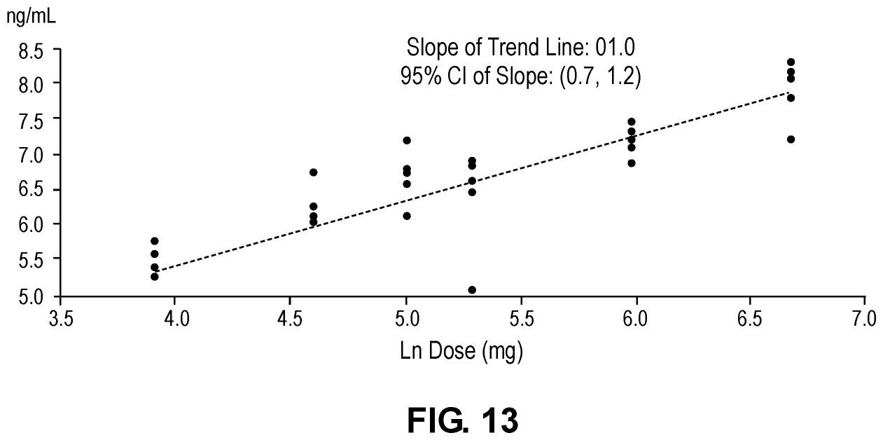

Results:
Participant demographics were well balanced by cohort (Table 1)
As can be seen in FIG. 11 and Table 2, plasma concentrations of Compound A increased in a dose-dependent manner
AUC_{inf} and Cₘₐₓ increased proportionally with dose (FIGS. 12 and 13; Tables 3 and 4)
Minimal food effect was observed following 100 mg dosing (Table 4)
Compound A was generally safe and well tolerated across all 6 dose cohorts:
29 TEAEs were observed, all TEAEs were mild (grade 1)
Top 3 most common TEAEs were headache, upper respiratory tract infection, and lightheadedness (2 incidences for each TEAE, respectively)
No drug-related TEAEs and no serious AEs (SAES)
TEAEs were equally distributed across all cohorts

TABLE 1
Participant Demographics (n = 48)

| | | Dose (mg) | | | | | | |
|---|---|---|---|---|---|---|---|---|
| Characteristics | | 50 | 100 | 150 | 200 | 400 | 800 | All |
| Age, Years | Mean (SD) | 27(5) | 26(5) | 25(4) | 17(11) | 27(6) | 28(11) | 25(8) |
| Race, | Caucasian | 38(3) | 88(7) | 63(5) | 50(4) | 75(6) | 50(4) | 60(29) |
| % (n) | Others | 63(5) | 13(1) | 38(3) | 50(4) | 25(2) | 50(4) | 40(19) |

TABLE 2

| Dose | Mean C₂₄ | % CV |
|---|---|---|
| 50 | 19.3 | 31.7 |
| 100 | 27.7 | 42.5 |
| 150 | 83.6 | 55.8 |
| 200 | 69.2 | 38.9 |
| 400 | 124.0 | 26.3 |
| 800 | 274.0 | 46.9 |

TABLE 3

| Dose | Mean AUC_{inf} | % CV |
|---|---|---|
| 50 | 2056 | 20.5 |
| 100 | 3325 | 47.3 |
| 150 | 8569 | 37.9 |
| 200 | 8382 | 30.2 |
| 400 | 14010 | 21.1 |
| 800 | 30180 | 35.7 |

TABLE 4

| Dose | Mean Cₘₐₓ | % CV |
|---|---|---|
| 50 | 244 | 20.1 |
| 100 | 449 | 59.3 |
| 150 | 848 | 33.9 |
| 200 | 693 | 43.0 |
| 400 | 1351 | 18.8 |
| 800 | 3015 | 32.0 |

TABLE 5
Mean (% CV) PK Parameters of Compound A by Dose -
Fasted Population (n = 36, 6 in Each Dose Cohort)

| | Dose (mg) | | | | | |
|---|---|---|---|---|---|---|
| Parameter | 50 | 100 | 150 | 200* | 400 | 800 |
| AUC_{inf} | 2056 (20.5) | 3325 (47.3) | 8569 (37.9) | 8382 (30.2) | 14010 (21.1) | 30180 (35.7) |
| (ng*hr/mL) | | | | | | |
| Cₘₐₓ | 244 (20.1) | 449 (59.3) | 848 (33.9) | 693 (43.0) | 1351 (18.8) | 3015 (32.0) |
| (ng/mL) | | | | | | |
| Tₘₐₓ | 1.8 (43.3) | 2.8 (109.7) | 1.9 (19.6) | 2.3 (50.2) | 2.4 (57.7) | 2.9 (33.3) |
| (Hours) | | | | | | |
| Half-Life | 6.5 (24.1) | 8.0 (33.0) | 9.7 (15.4) | 9.8 (11.3) | 9.5 (9.9) | 9.0 (9.9) |
| (Hours) | | | | | | |

*5 subjects in 200 mg cohort had data for AUC_{inf} and Cₘₐₓ

TABLE 6
Geometric Mean (Geometric % CV) AUC_{inf} and
Cₘₐₓ Following Compound A 100 mg - Fasted vs.
Fed Conditions (n = 6 Under Each Condition)

| Parameter | Fasted | Fed | % Ratio |
|---|---|---|---|
| AUC_{inf} (ng*hr/mL) | 2573 (135.1) | 3866 (16.1) | 150.3 |
| Cₘₐₓ (ng/mL) | 297 (236.1) | 382 (35.8) | 128.5 |

Compound A systemic exposure increased in a dose dependent manner and was largely proportional to dose. PK results showed low to moderate between-subject variability. Compound A PK after a high fat, high caloric meal was similar to fasting conditions. Once-daily dosing of Compound A was generally well tolerated with no moderate or severe TEAEs, no drug-related TEAEs, no SAEs, and no dose limiting toxicity. Results demonstrate a PK profile as predicted and that Compound A is a potent, safe, oral plasma kallikrein inhibitor for the prophylactic treatment of hereditary angioedema (HAE).

Although the invention has been described with reference to the above examples, it will be understood that modifications and variations are encompassed within the spirit and scope of the invention. Accordingly, the invention is limited only by the following claims.

What is claimed is:

1. A method of treating angioedema in a patient in need thereof, comprising administering to the patient a composition comprising N-((6-amino-2,4-dimethylpyridin-3-yl)methyl)-2-((3-chloroquinolin-6-yl)methyl)isonicotinamide, or a pharmaceutically acceptable salt thereof.

2. The method of claim 1, wherein the angioedema is hereditary angioedema.

3. The method of claim 1 or 2, wherein the composition is administered daily.

4. The method of claim 3, wherein the composition is administered once or twice per day.

5. The method of any one of claims 1-4, wherein the composition is administered orally.

6. The method of any one claims 1-5, wherein the N-((6-amino-2,4-dimethylpyridin-3-yl)methyl)-2-((3-chloroquinolin-6-yl)methyl)isonicotinamide is administered in an amount of from about 300 mg/day to about 800 mg/day.

7. The method of claim 6, wherein the N-((6-amino-2,4-dimethylpyridin-3-yl)methyl)-2-((3-chloroquinolin-6-yl)methyl)isonicotinamide is administered in an amount of about 300 mg/day, about 350 mg/day, about 400 mg/day, about 450 mg/day, about 500 mg/day, about 600 mg/day, about 650 mg/day, about 700 mg/day, about 750 mg/day, or about 800 mg/day.

8. The method of claim 6, wherein the N-((6-amino-2,4-dimethylpyridin-3-yl)methyl)-2-((3-chloroquinolin-6-yl)methyl)isonicotinamide is administered in an amount of about 300 mg.

9. The method of claim 8, wherein the N-((6-amino-2,4-dimethylpyridin-3-yl)methyl)-2-((3-chloroquinolin-6-yl)methyl)isonicotinamide is administered twice per day.

10. The method of claim 6, wherein the N-((6-amino-2,4-dimethylpyridin-3-yl)methyl)-2-((3-chloroquinolin-6-yl)methyl)isonicotinamide is administered in an amount of about 400 mg/day, about 450 mg/day, about 500 mg/day, about 550 mg/day, about 600 mg/day, about 650 mg/day, about 700 mg/day, about 750 mg/day, or about 800 mg/day.

11. The method of claim 10, wherein the N-((6-amino-2,4-dimethylpyridin-3-yl)methyl)-2-((3-chloroquinolin-6-yl)methyl)isonicotinamide is administered in an amount of about 400 mg/day.

12. The method of claim 10, wherein the N-((6-amino-2,4-dimethylpyridin-3-yl)methyl)-2-((3-chloroquinolin-6-yl)methyl)isonicotinamide is administered in an amount of about 450 mg/day.

13. The method of claim 10, wherein the N-((6-amino-2,4-dimethylpyridin-3-yl)methyl)-2-((3-chloroquinolin-6-yl)methyl)isonicotinamide is administered in an amount of about 500 mg/day.

14. The method of claim 10, wherein the N-((6-amino-2,4-dimethylpyridin-3-yl)methyl)-2-((3-chloroquinolin-6-yl)methyl)isonicotinamide is administered in an amount of about 550 mg/day.

15. The method of claim 10, wherein the N-((6-amino-2,4-dimethylpyridin-3-yl)methyl)-2-((3-chloroquinolin-6-yl)methyl)isonicotinamide is administered in an amount of about 600 mg/day.

16. The method of claim 10, wherein the N-((6-amino-2,4-dimethylpyridin-3-yl)methyl)-2-((3-chloroquinolin-6-yl)methyl)isonicotinamide is administered in an amount of about 650 mg/day.

17. The method of claim 10, wherein the N-((6-amino-2,4-dimethylpyridin-3-yl)methyl)-2-((3-chloroquinolin-6-yl)methyl)isonicotinamide is administered in an amount of about 700 mg/day.

18. The method of claim 10, wherein the N-((6-amino-2,4-dimethylpyridin-3-yl)methyl)-2-((3-chloroquinolin-6-yl)methyl)isonicotinamide is administered in an amount of about 750 mg/day.

19. The method of claim 10, wherein the N-((6-amino-2,4-dimethylpyridin-3-yl)methyl)-2-((3-chloroquinolin-6-yl)methyl)isonicotinamide is administered in an amount of about 800 mg/day.

20. The method of any one of claims 10-18, wherein the N-((6-amino-2,4-dimethylpyridin-3-yl)methyl)-2-((3-chloroquinolin-6-yl)methyl)isonicotinamide is administered once per day.

21. The method of any one claims 1-20, wherein the composition is formulated for immediate release.

22. The method of any one claims 1-21, wherein the composition is formulated for as a tablet or capsule.

23. The method of any one claims 1-22, wherein the composition further comprises at least one pharmaceutically acceptable excipient.
